(19) United States
(12) Patent Application Publication
(10) Pub. No.: US 2019/0292522 A1
(43) Pub. Date: Sep. 26, 2019

(54) NOTCH ACTIVATION ENHANCES MESENCHYMAL STEM CELL SHEET OSTEOGENIC POTENTIAL BY INHIBITION OF CELLULAR SENESCENCE

(71) Applicant: Board of Supervisors of Louisiana State University and Agricultural and Mechanical College, Baton Rouge, LA (US)

(72) Inventor: Yufeng DONG, Shreveport, LA (US)

(21) Appl. No.: 16/264,635

(22) Filed: Jan. 31, 2019

Related U.S. Application Data
(60) Provisional application No. 62/624,975, filed on Feb. 1, 2018.

(51) Int. Cl. C12N 5/0775; C12N 5/00; A61P 19/00; A61L 27/38; A61L 27/54; A61K 35/28

(52) U.S. Cl. CPC C12N 5/0663; C12N 5/0062; A61P 19/00; A61L 27/3847; C12N 2510/00; A61L 27/3834; A61K 35/28; A61L 2430/02; C12N 2513/00; A61L 27/54

(57) ABSTRACT

A method of retaining multi-potency in stem cells comprising one of reducing p16RNA expression and increasing Hes1 expression. A therapeutic comprising a high density sheet of stem cells wherein the stem cells are Notch activated. A method of treating bone injury comprising administering stem cells that are Notch activated. Notch is activated by adding Jagged1.

### CROSS REFERENCE TO RELATED APPLICATIONS/PRIORITY

The present invention claims priority to United States Provisional Patent Application No. 62/624,975 filed Feb. 1, 2018, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### BACKGROUND

MSC sheet-based applications to skeletal regeneration and repair in the clinic currently require large numbers of functional cells, and high density cell sheet culture often induces rapid stem cell aging process accompanied by loss of proliferative and differentiation capacity. To maintain stem cell phenotype in cultures and avoid cell sheet culture-induced cell aging has become necessary, though achieving it has been a seemingly irresolvable challenge for medical technology.

### SUMMARY

Wherefore, it is an object of the present invention to overcome the above mentioned shortcomings and drawbacks associated with the current technology.

The presently claimed invention relates to methods of retaining multi-potency in stem cells comprising one of reducing p16RNA expression and increasing Hes1 expression. According to further embodiments p16RNA expression is reduced by activating Notch. According to further embodiments Hes1 expression is increased by activating Notch. According to further embodiments one of p16RNA expression is reduced by activating Notch and Hes1 expression is increased by activating Notch. According to further embodiments Notch is activated by adding Jagged1. According to further embodiments the stem cells are mesenchymal stem cells (MSC). According to further embodiments the stem cells are in a high density sheet.

The presently claimed invention further relates to a therapeutic comprising a high density sheet of stem cells, wherein the stem cells are Notch activated. According to further embodiments Notch is activated with Jagged1.

The presently claimed invention further relates to a method of treating bone injury comprising administering stem cells that are Notch activated. According to further embodiments the stem cells are mesenchymal stem cells (MSC). According to further embodiments the stem cells are in a high density sheet. According to further embodiments the stem cells are long term cultured cell sheet. According to further embodiments the stem cells are cultured at least 5 days. According to further embodiments the stem cells are part of an allograft administered to the bone injury of the patient. According to further embodiments the stem cells are mesenchymal stem cells (MSC), the stem cells are in a high density sheet, the stem cells have been cultured for at least 5 days, and the stem cells are part of an allograft administered to the bone injury of the patient.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIGS. 1A to 1F show that a Cell sheet culture induces differentiation in mesenchymal stem cells (MSCs). (FIG. 1A) The effect of sheet culture on MSC morphologic change was examined in bright field (upper). Scale bar represents 100 μm. ALP staining revealed MSC differentiation (lower). (FIG. 1B) Representative flow cytometry histograms showing CD105 expression in MSCs from control (80% confluence), day1 cell sheet, and day5 cell sheet. (FIG. 1C) Quantification of the CD105 subpopulations in total MSCs from flow cytometry data. (FIG. 1D) BrdU ELISA to monitor proliferation rate in MSCs from control (Co) (80% confluence), day1 cell sheet (Day1), and day5 cell sheet (Day5). (FIG. 1E) The percent of apoptotic cells was measured by Caspase-3/7 assay in MSCs from control (80% confluence), day1 sheet, and day5 sheet. (FIG. 1F) Gene expression for MSC differentiation marker (ALP), cell cycle regulator (CCND1, p16, p21) in control, day1 sheet, and day5 sheet was measured by quantitative polymerase chain reaction. Results are mean±S.D. All experiments were performed in triplicate. *P<0.05 versus control cells.

FIGS. 2A to 2D show the effects of JAG1 on MSC senescence and cell cycle distribution in sheet cultures. (FIG. 2A) SA-β-gal staining was performed to determine the extent of senescence in 1- and 5-day sheet cultures with JAG1 or IgG treatment. Scale bar represents 100 μm. (FIG. 2B) SA-β-gal positive cells were quantitated by ImageJ. (FIG. 2C) Representative flow cytometry graphs of cell cycle analysis. (FIG. 2D) The percentage of cells in G0, G1, and S/G2/M from 1- and 5-day sheet cultures. Results are mean±S.D. All experiments were performed in triplicate. *P<0.05 versus IgG day1 control cells; ^{#}P<0.05 versus IgG day5 control cells.

FIGS. 3A to 3D show an mRNA expression profile of MSC changes in cell sheet cultures. Differential expression of cell-cycle-related genes was verified by quantitative RT-PCR. (FIG. 3A) CCND1 gene expression increased by JAG1 in 1-day cell sheet cultures. (FIG. 3B) p16 gene expression decreased by JAG1 in both 1- and 5-day cell sheet cultures. (FIG. 3C) p21 gene expression increased from day 1 to day 5 in control cell sheet cultures but was not altered by JAG1 treatment. (FIG. 3D) Hes1 gene expression decreased from day 1 to day 5 in control cell sheet cultures, and JAG1 significantly induced Hes1 expression in both 1- and 5-day cultures. Results are mean±S.D. All experiments were performed in triplicate. *P<0.05 versus IgG day-1 control cells; ^{#}P<0.05 versus IgG day5 control cells.

FIGS. 4A-4F show Hes1 is required for JAG1-induced inhibition of MSC aging. (FIG. 4A) Bright field image showed a high infection efficiency in lentiviral GFP control MSCs after 24 h of culture. Scale bar represents 50 μm. (FIG. 4B) Western blot analysis confirmed the decrease of Hes1 protein levels at day 5 in Hes1 knockdown MSCs, even with JAG1 treatment. (FIG. 4C) SA-β-gal staining was performed to determine the extent of senescence in shHes1-infected MSCs at day 5 in cell sheet culture with JAG1 treatment. Scale bar represents 100 μm. (FIG. 4D) SA-β-gal positive cells in cell sheet cultures were quantitated by ImageJ. (FIG. 4E) Quantitative reverse transcriptase polymerase chain reaction (qRT-PCR) data showed that the decreased expression of p16 by JAG1 was rescued by knockdown of Hes1 in MSCs at day5 in sheet cultures. No changes were observed in p21 expression in 5-day sheet cultures. (FIG. 4F) Western blot data showed a similar expression pattern of p16 and p21 protein level to RNA expression in JAG1 treated wild type MSCs and Hes1 deficient MSCs. Results are mean±S.D. All experiments were performed in triplicate. *P<0.05 versus IgG control cells.

FIGS. 5A-5E show 5-day MSC sheets enhance bone callus formation and improve the biomechanical torsional properties of grafted femurs at 6-weeks post-surgery. (FIG. 5A) The left panels are representative x-ray images of allografts alone, allografts wrapped with IgG cultured 5-day sheets (Day5 sheets), and allografts wrapped with Notch activation cultured 5-day MSC sheets (Day5 sheet/JAG1) at 6-weeks post-surgery. Right panels show the histological sections stained with AB/H/OG. Scale bars=400 μm. Bone callus is marked with black triangles and cartilaginous callus is marked with black arrows. (FIG. 5B) Representative Micro-CT volumetric rendering of the grafted femurs with 5-day IgG or JAG1 MSC sheets at 6-weeks post-surgery. Allograft alone was used as the control. (FIG. 5C) Quantification of bone volume/total tissue volume (BV/TV) from the mineralized calluses of each group. (*, P<0.05 compared with allograft alone; #, P<0.05 compared with allograft+Day5 sheets). (FIG. 5D) Maximal torque and (FIG. 5E) Torsional rigidity of grafted femurs from each group was retrieved upon mice (n=6) sacrifice at 6-weeks post-surgery. Data presented as mean±standard deviation. *P<0.05 compared with allograft alone; ^{#}P<0.05 compared with allograft+Day5 sheets.

FIG. 6 is a possible model of Notch signaling transduction in regulation of MSC in vitro aging. Activations of Notch signaling by Jagged1 (JAG1) in MSC sheet culture stimulates cell proliferation and inhibits aging. Notch signaling includes sequential stimulation of a signaling cascade involving Notch intracellular domain (NICD) and Hes1. NICD induces the expression of Hes1, which may lead to activation of Cyclin D1 (CCND1) and inhibition of p16, leading to the delay of MSC aging in cultures.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

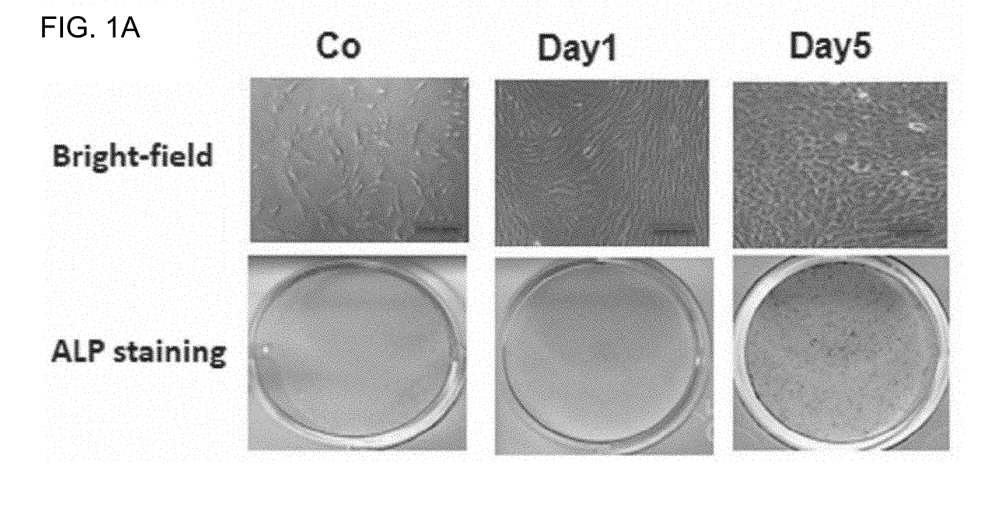

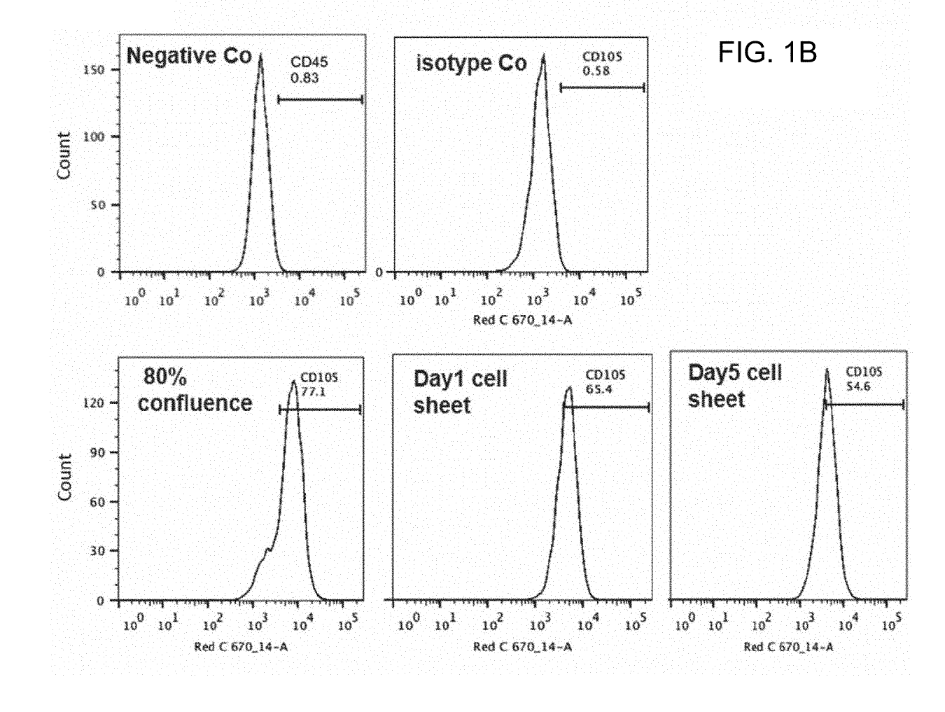

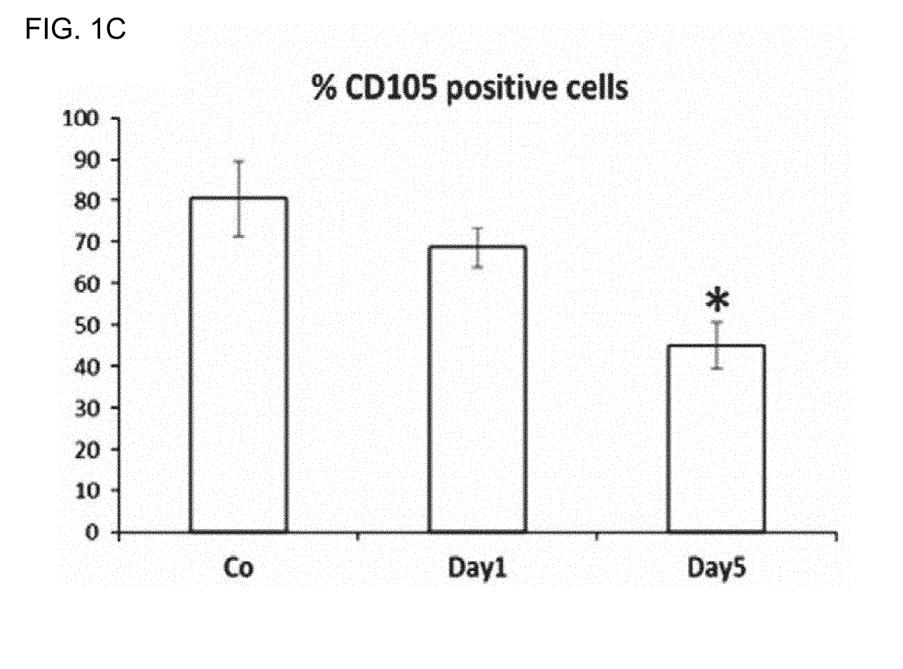

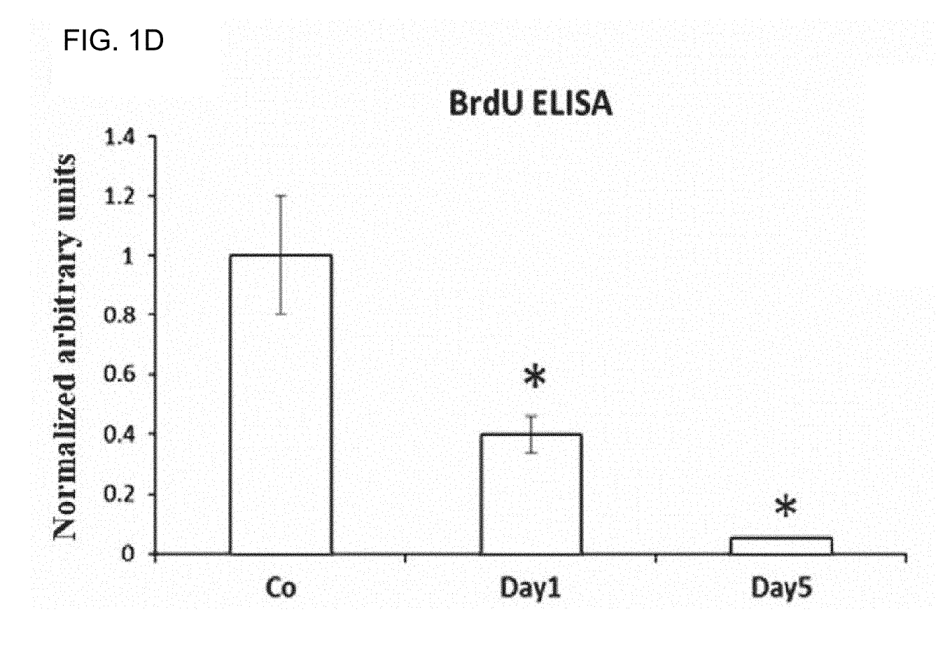

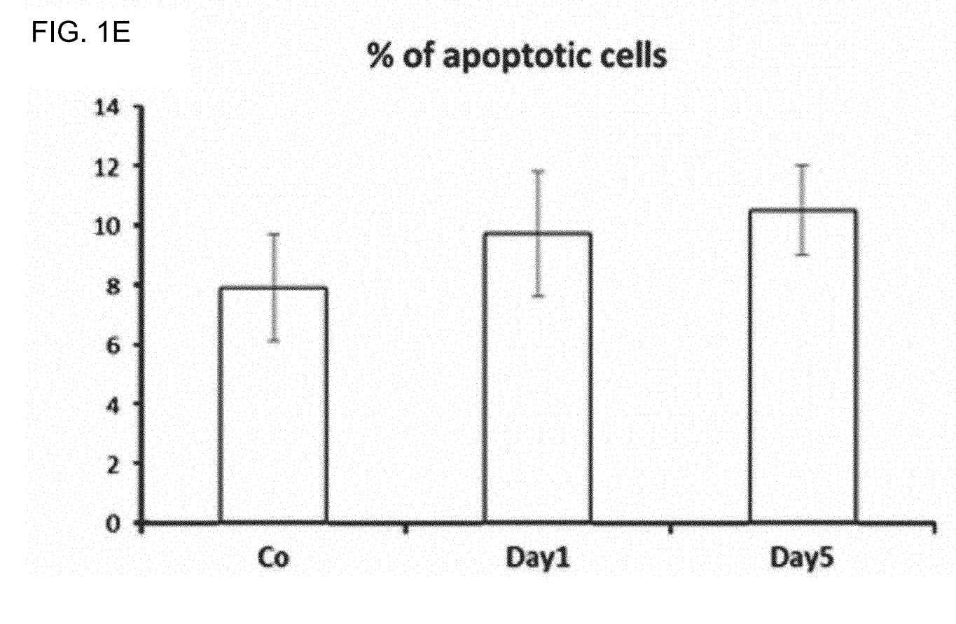

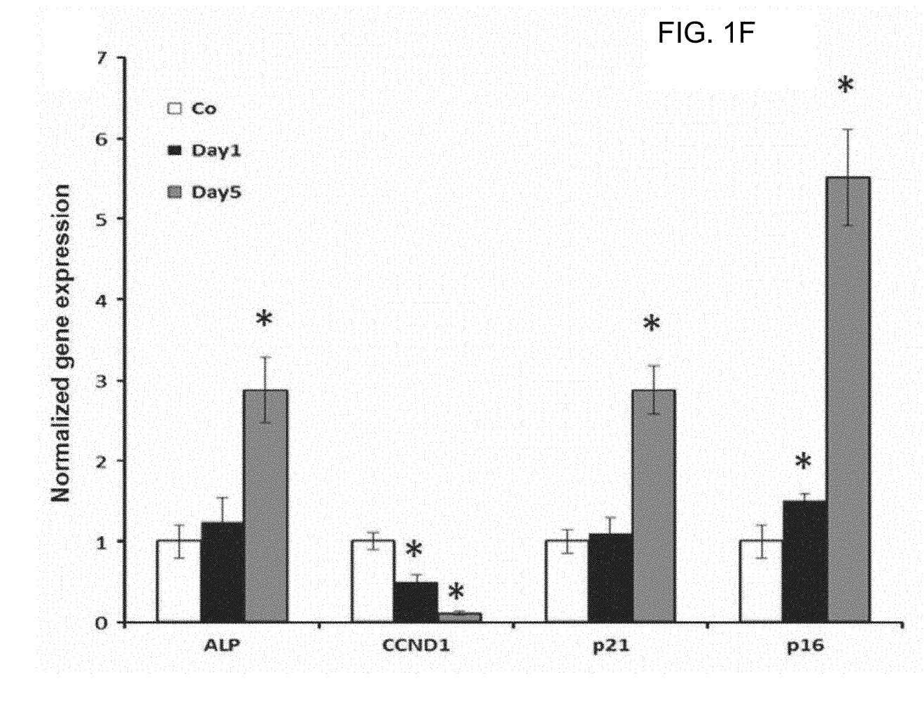

Turning now to FIGS. 1 to 6, a brief description concerning the various components of the present invention will now be briefly discussed.

The inventors' previous studies have confirmed the therapeutic effects of mesenchymal stem cell (MSC) monolayer sheet transplantation on allograft repair. A limiting factor in their application is the loss of MSC multi-potency as a result of high density sheet culture-induced senescence. In the study disclosed herein, the inventors tested whether Notch activation could be used to prevent or delay sheet culture-induced cell aging. The inventors' results show that, during in vitro long-term (5-day) cell sheet culture, MSCs progressively lose their progenitor characteristics. In contrast, Notch activation by Jagged1 in MSC sheet culture showed reduced cellular senescence and cell cycle arrest compared with control MSCs without Notch activation. Importantly, knockdown of Notch target gene Hes1 totally blocked the inhibition effect of Jagged1 on cellular senescence. Finally, the in vivo allograft transplantation data showed a significant enhanced callus formation and biomechanical properties in Notch activation cultured long-term sheet groups when compared with long-term cultured sheet without Notch activation. The inventors' results suggest that Notch activation by Jagged1 could be used to overcome the stem cell aging caused by high density sheet culture, thereby increasing the therapeutic potential of MSC sheets for tissue regeneration.

Mesenchymal stem cells (MSCs) provide a promising cell source for bone tissue regeneration, which is under investigation in several clinical trials. However, appropriate delivery and retention of MSCs or osteogenic cells during therapeutic strategies for skeletal repair remain extremely challenging. While conventional in vivo delivery methods (single-cell suspensions, injection, seeding to or mixed with scaffolds) have demonstrated some success, problems still remain, including rapid cell diffusion, uneven cell distribution across the scaffolds, and weak adhesion of MSCs seeded to the scaffolds, causing easy detachment from graft following in vivo transplantation. To overcome these problems, stem cell sheets generated in temperature-responsive culture dishes have been used by the inventors group to enhance allograft healing during repair of large bone defects. Since cell sheet culture often induces stem cell aging, characterized by increased senescence and a rise in the levels of the cell cycle inhibitors p16 and p21, only short-term (1-day) cultured stem cell monolayer sheets have been tested in our mouse allograft healing model.

The inventors are aware of various signaling factors implicated in the regulation of MSC maintenance and expansion, including FGF, Wnt and TGF pathways. Recently, the inventors identified the Notch signaling pathway as an important regulator of MSC proliferation and differentiation using an in vivo mouse model and successfully utilized Jagged1 (JAG1)-mediated Notch activation to rapidly expand MSCs in regular cell cultures. In the present disclosure the inventors undertook a study to further determine if Notch signaling could be used to prevent or delay rapid cell aging induced by long-term (5-day) sheet culture. In addition, prolonged culture provides extra time for a surgeon to make a flexible transplantation schedule, which avoids any unnecessary MSC discarding.

The Notch signaling pathway is an evolutionarily conserved signaling system that regulates cell proliferation, differentiation, and fate determination in both embryonic and adult organs. In mammals, Notch signaling is initiated when one of the 11 Notch ligands (Jagged1-2; DLL1,3,4; DLK1-2; MAGP1-2; DNER; and NB3) activates a single-pass transmembrane cell surface Notch receptor (Notch1-4), resulting in a series of receptor cleavage events (S2 and S3) that release the Notch intracellular domain (NICD) into the cytoplasm via ADAM and Presenilin (PSEN1/2) protease activities. NICD translocates to the nucleus and binds the transcriptional regulators, RBPjK and MAML, creating a transcriptionally active complex. Upon activation, NICD-RBPjK-MAML ternary complexes drive the expression of downstream target genes, such as the Hes and Hey family of bHLH transcription factors. This axis of classic Notch signaling is often referred to as the RBPjK-dependent canonical Notch pathway. Notch function in embryonic stem cells, somite progenitors, and neural progenitors has shown that ultradian oscillations of Notch and Hes signals appeared to be required for the normal development and maintenance of various cell lineages and tissues. A previous study of the inventors identified JAG1-Notch2-Hes1 as the dominant Notch molecules in MSCs. The inventors further tested whether the Notch ligand JAG1 could be utilized to inhibit senescence in long-term sheet culture, and finally enhance their therapeutic effect in vivo.

### Results

Increased MSC aging in response to cell sheet culture: Long-term culture sometime is needed in clinical settings. However, cell aging and differentiation can be accelerated due to the high degree of cell-cell contact in long-term culture. To study the effects of cell sheet culture on MSC aging, the inventors compared MSCs from 1-day and 5-day sheets with control MSCs from 80% confluence culture. Cell morphology changes were first observed in a bright field. Control and 1-day sheet MSCs showed a similar fibroblast-like appearance with few cytoplasmic expansions, while MSCs from 5-day sheets were found to be significantly larger and widely spread out (FIG. 1A upper panel). These findings suggest sheet-culture induces cell differentiation and senescence in MSC culture. The enhanced cell differentiation was further confirmed by alkaline phosphatase (ALP) staining, which showed increased ALP activity in 5-day MSC sheets compared with control and 1-day MSC sheets (FIG. 1A lower panel).

The effects of biologic aging on immunophenotypic change of MSCs were analyzed by flow cytometry for cell surface antigen CD105, a typical marker for less differentiated stromal cells. The percentage of CD105 positive cells was 79.8±8.6% in 80% confluence MSCs, 70.7±3.5% in 1-day sheet MSCs, and 45.5±5.2% in 5-day sheet MSCs (FIGS. 1B and 1C) suggesting a progressive loss of stromal cell population in cell sheet cultures. To eliminate lymphocyte contamination, lymphocyte cell surface marker CD45 was also analyzed and <1% of the cells were positive for this marker. In addition, the appropriately matched isotype control (IgG-FITC) was used to set the gate for the CD105-positive population (FIG. 1B).

To assess sheet culture-dependent changes in MSC proliferation, the inventors performed a BrdU-labeling based cellular proliferation assay. MSCs from the 5-day sheet culture exhibited a markedly reduced proliferation rate compared with control MSCs and 1-day sheet cultures (FIG. 1D). Interestingly, cell apoptosis was not found to be significantly increased in 5-day sheet culture compared with control and 1-day cultures when assayed by caspase 3/7 activity (FIG. 1E) indicating that apoptosis was not involved in this aging process. To further validate this age-related alteration in cell sheet cultures, markers associated with the aging phenotype of stromal cells (e.g., the cell-cycle regulation protein cyclinD1, p16, and p21) were analyzed by real-time PCR (FIG. 1F). As expected, cell proliferation marker cyclinD1 (CCND1) expression was significantly reduced in 5-day cultures compared with control and 1-day cultures, whereas the expression of cell cycle inhibitors p16 and p21 was significantly up-regulated in 5-day sheet cultures. Consistent with ALP staining, the RNA expression of MSC spontaneous differentiation marker ALP was also significantly induced in 5-day cultures compared with control and 1-day cultures, further confirming progressive cell aging differentiation in long-term cell sheet cultures.

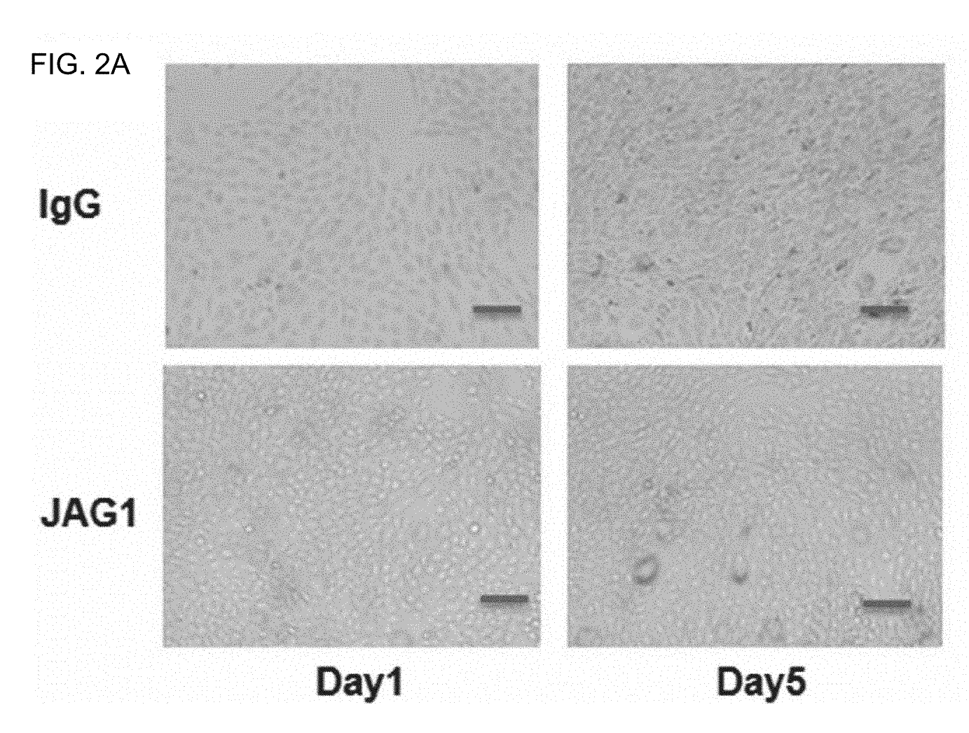

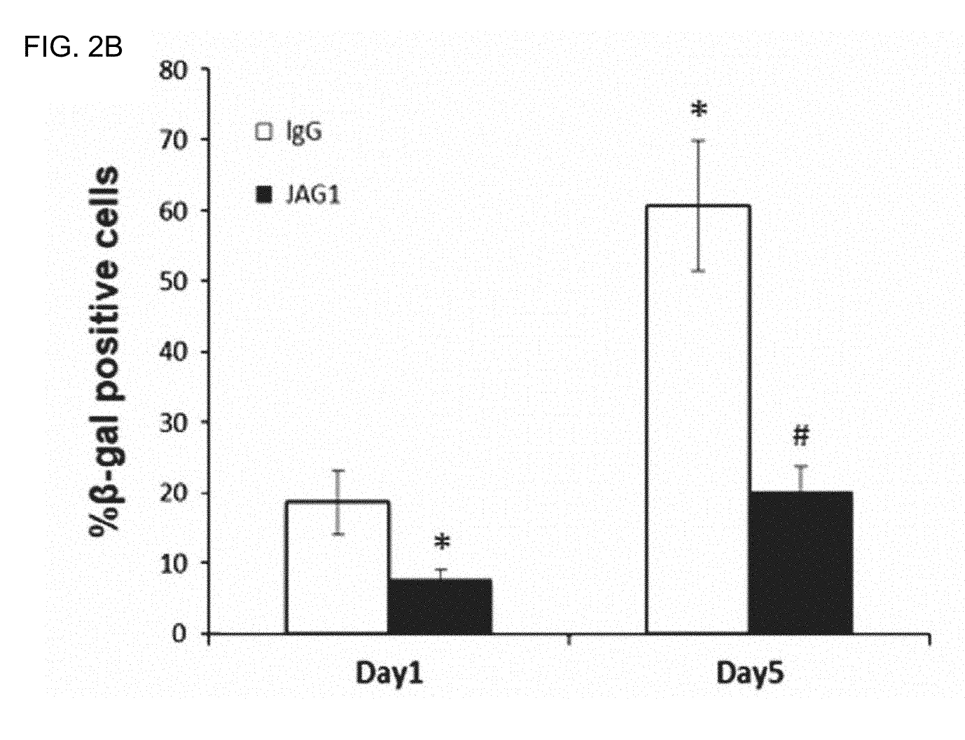

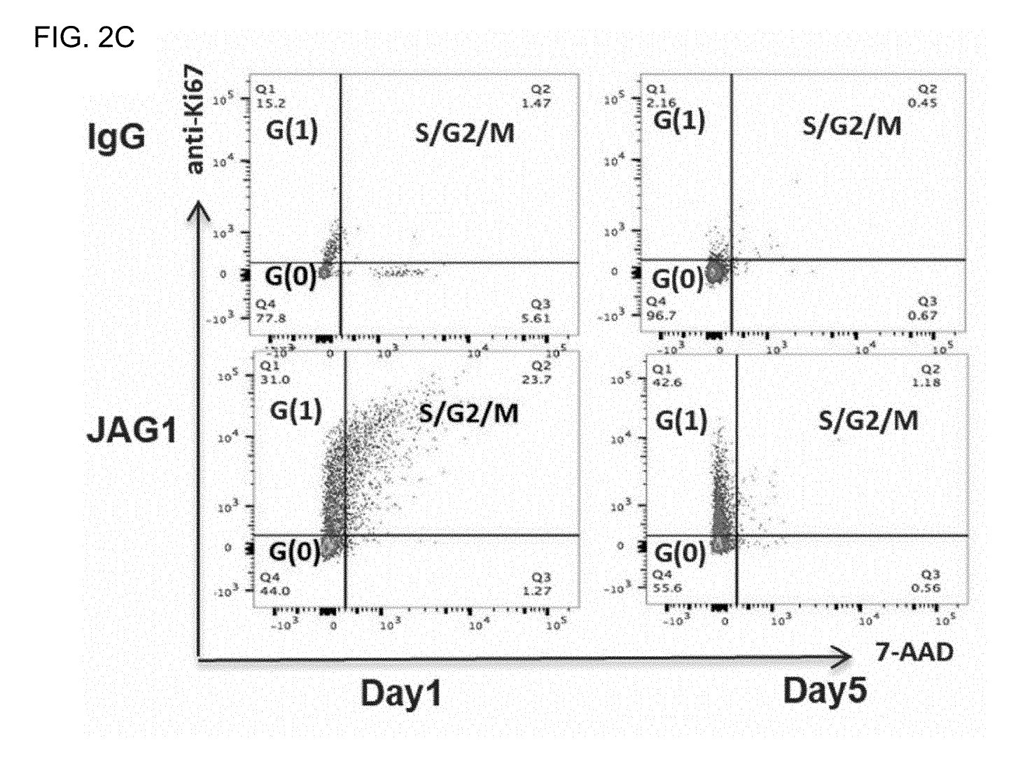

Decreased MSC senescence within JAG1-coated plates: Cellular senescence is a phenomenon of cell aging, which can be detected by monitoring senescence associated β-galactosidase activity (SA-β-gal). To monitor the senescence status of MSCs during sheet culture, the inventors performed β-galactosidase staining in 1-day and 5-day cultures. The data showed that the percentage of MSCs positive for SA-β-gal increased with time, from 19.25±4.6% at day 1 to 60.48±8.29% at day 5 (FIGS. 2A and 2B), indicating a progressive cell senescence in long-term sheet cultures. Since the inventors' previous study showed that Notch activation by JAG1 enhances stem cell maintenances in vivo and in vitro, the inventors further monitored cellular senescence in long-term sheet culture on JAG1 ligand coated plates. As the inventors predicted, activation of Notch signaling by JAG1 led to a reduced cell senescence in both 1- and 5-day sheet cultures, as demonstrated by decreased frequencies of SA-β-gal positive cells (8.25±1.5% and 20.5±4.2%, respectively) when compared with MSCs from IgG control groups (P<0.05) (FIGS. 2A and 2B).

Cell cycle regulation is required for MSCs to remain in an undifferentiated state and to exit the cell cycle during cellular senescence progression. To further determine the effect of JAG1 on cell cycle distribution, the inventors performed flow cytometric analysis using MSCs from 1-day and 5-day sheet cultures after labeling them with 7-AAD and Ki67. The percentages of cells in the G0, G1, and S/G2/M phases of the cell cycle were then calculated. In IgG control groups, MSCs in 1-day and 5-day cultures possessed a high percentage of cells in the G0 phase (80.96±5.5% and 97.5±5.4%, respectively) due to sheet culture-induced cell cycle arrest. By comparison, the G0 phase cells in JAG1 groups was 45.2±2.8% and 57.5±8.2% in the 1-day and 5-day cultures respectively, and significantly lower than those in the IgG groups (FIGS. 2C and 2D). These data demonstrate an increased fraction of cells arrested in the G0 phase during long-term sheet cultures. Collectively, these studies indicate that Notch activation could reduce senescence progression by promoting more cells into G1 and S/G2/M phases, and supporting the inventors' hypothesis that activation of Notch signaling delays cell aging in sheet cultures.

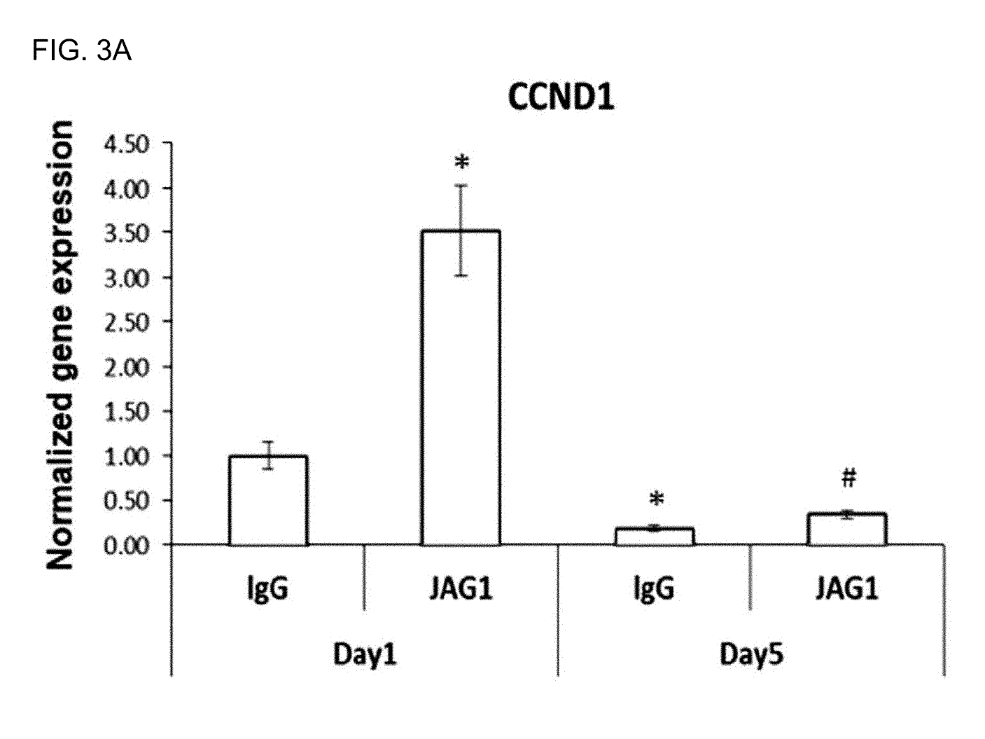

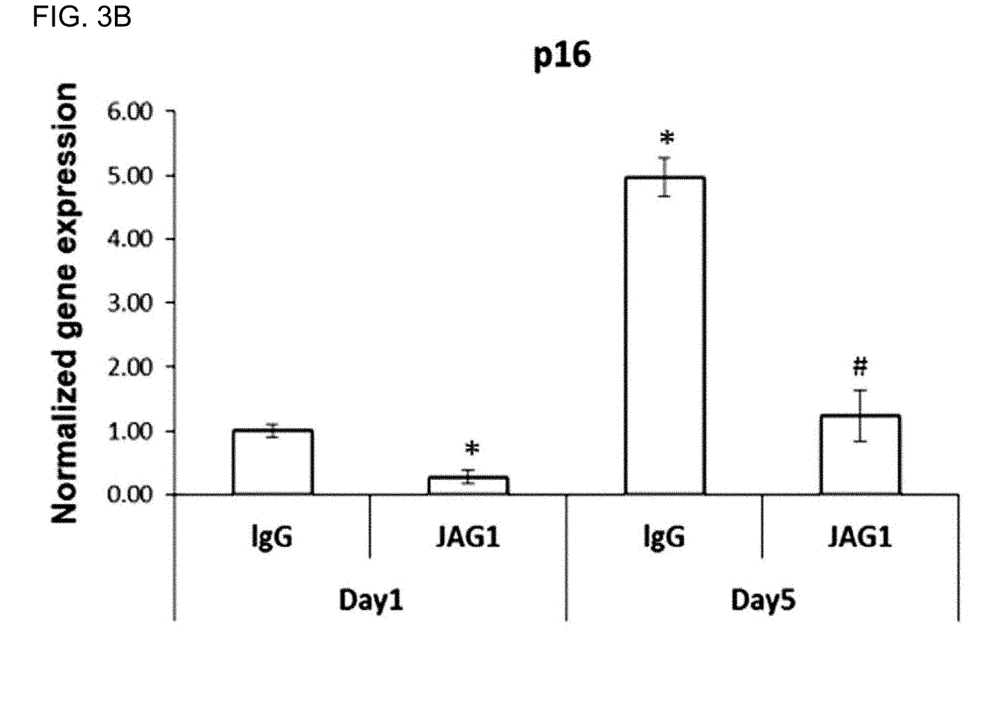

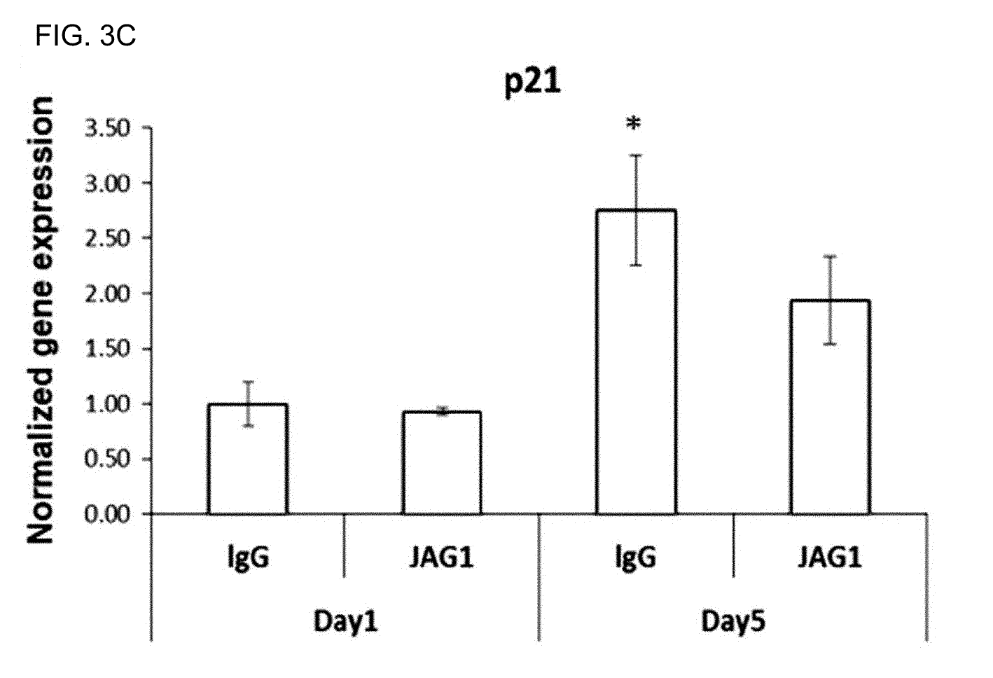

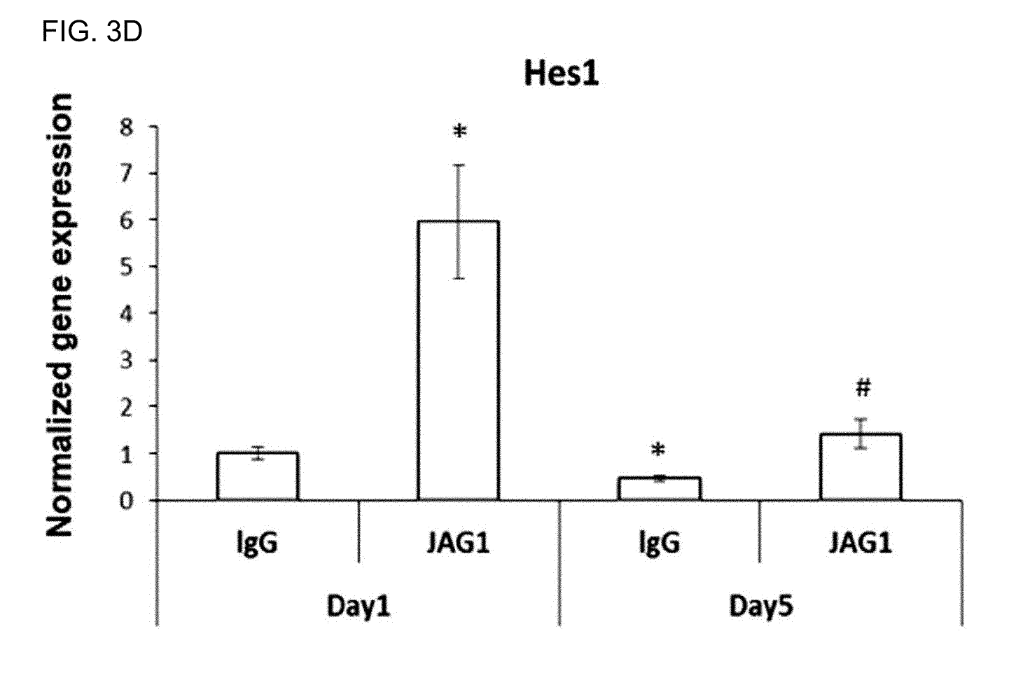

To obtain insight into the anti-aging effects of JAG1 in MSCs at the molecular level, the inventors monitored the expression of the proliferation marker CCND1 and the typical senescence markers, p16 and p21 in both short-term and long-term sheet cultures. Consistent with the inventors' previous finding, CCND1 expression was induced by JAG1 in 1-day cultures and no significant changes were observed in 5-day cultures compared with IgG controls (FIG. 3A). In the IgG control group, relative quantification by RT-PCR revealed an ˜5-fold up-regulation of p16 RNA (FIG. 3B) and a 2.7-fold up-regulation of p21 RNA in 5-day cultures compared with 1-day cultures (FIG. 3C), which suggests that these two factors act synergistically to induce MSC senescence. Interestingly, although both p16 and p21 play an important role in cellular senescence, only p16 expression was significantly reduced and maintained in lower levels in the JAG1 groups at both 1- and 5-day cultures. In contrast, JAG1 did not show a significant inhibition effect on p21 gene expression in both 1- and 5-day cultures (FIGS. 3B and 3C). These observations suggest that p16, not p21, is the major downstream factor responsible for the Notch-induced anti-aging effect in cell sheet cultures. Taken together, these results demonstrate that MSC aging induced by cell sheet culture can be remarkably reduced by activation of Notch signaling.

Since JAG1 effectively reversed MSC senescence in cell sheet culture, the inventors next investigated the possible Notch signaling downstream molecules involved in this process. Previously the inventors had determined that Notch signaling maintains and expands MSCs via a JAG1-Notch-Hes1 signaling axis during mouse skeletal development. Therefore, the inventors further measured Notch target Hes1 expression in 1- and 5-day sheet cultures. RT-PCR analysis of total RNA revealed a decreased expression of Hes1 from 1-day to 5-day cultures in control IgG-coated plates, while Hes1 expression at both time points was significantly increased by JAG1 (FIG. 3D). This suggests that canonical Notch target gene Hes1 is involved in this JAG1-mediated inhibition of MSC aging.

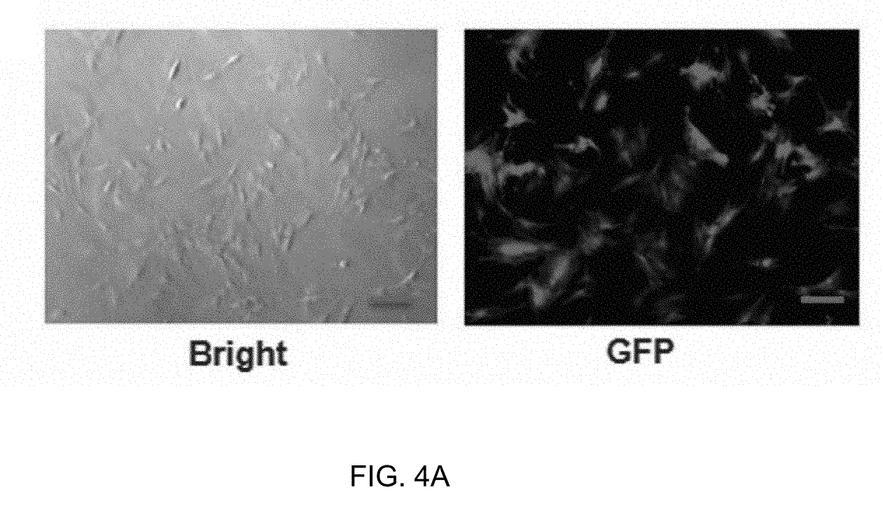

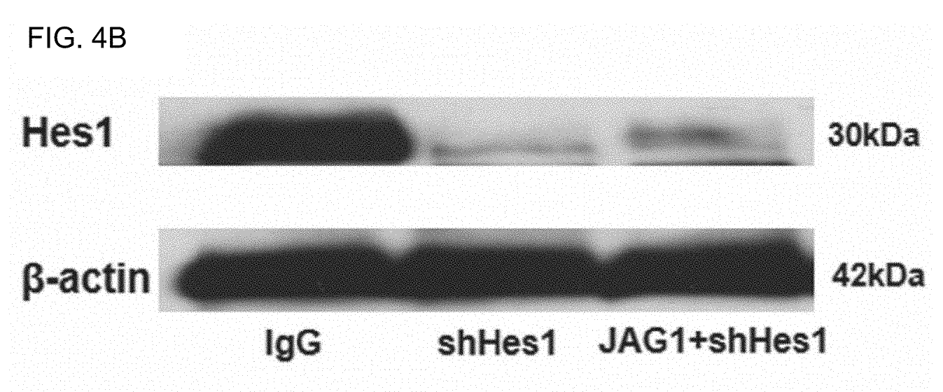

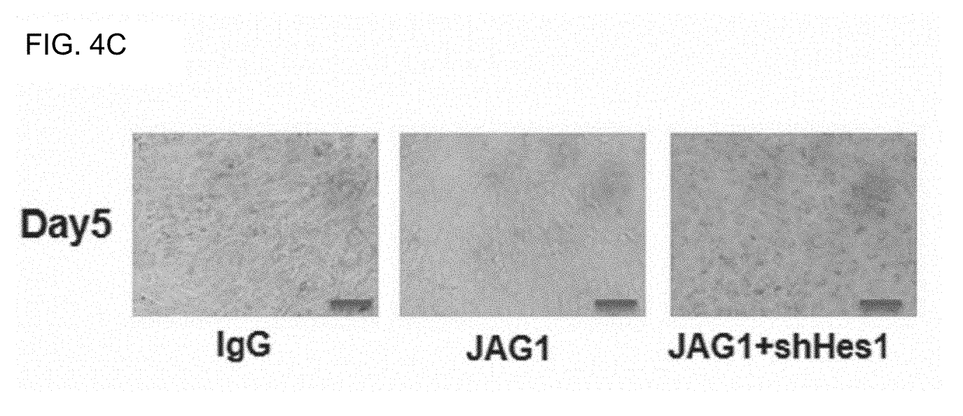

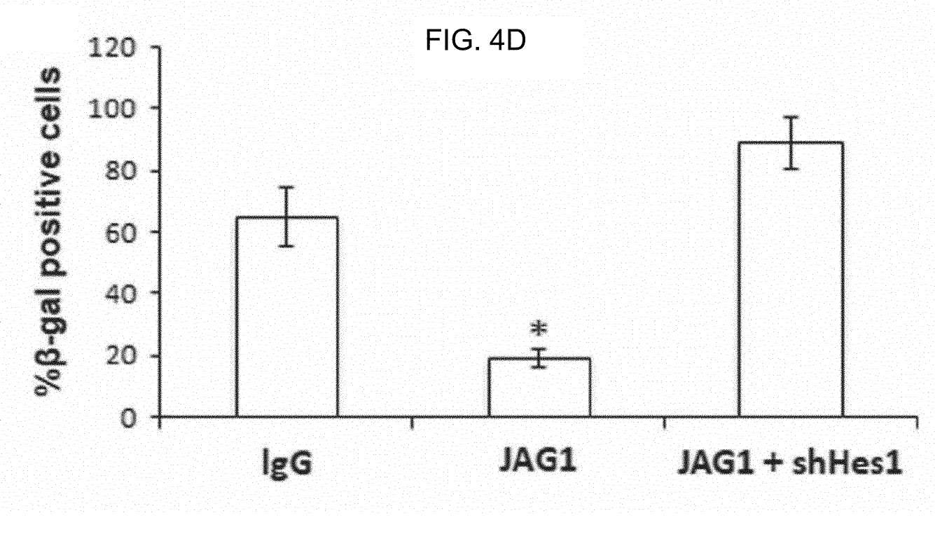

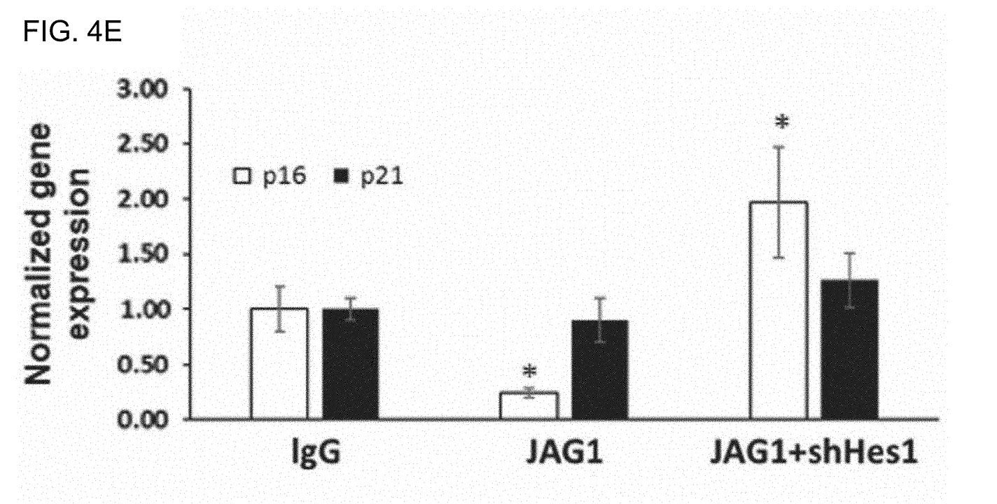

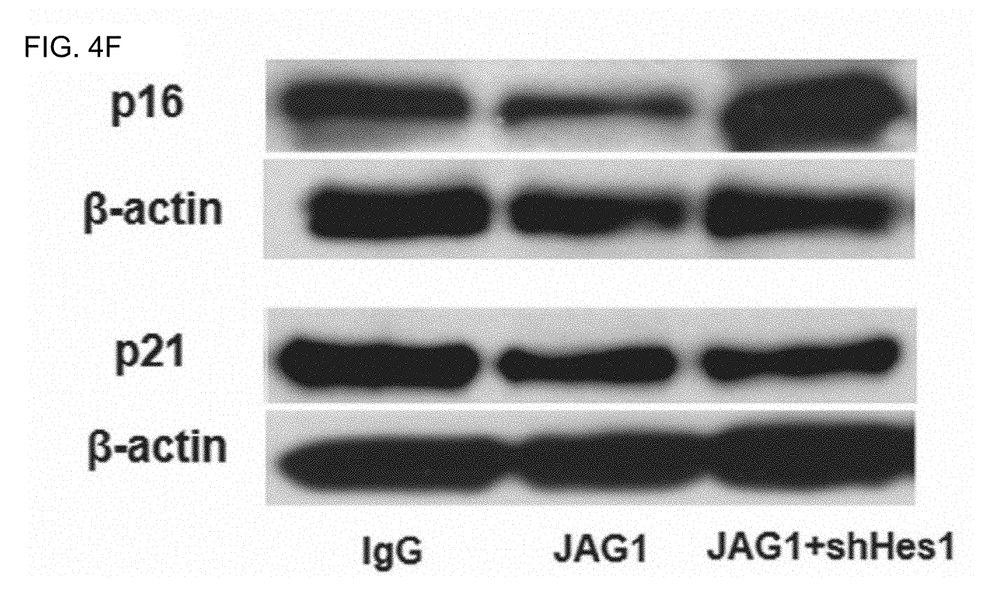

Knockdown of Hes1 expression blocks JAG1-mediated inhibition of MSC aging: To further test whether Hes1 is required in JAG1-mediated inhibition of cellular senescence, the inventors knocked down Hes1 expression using shRNA lentiviral particles in MSC sheet cultures. After 24 h of culture with lentivirus, GFP controls showed that 90% of MSCs were successfully infected by lentiviral particles (FIG. 4A). Western blot analysis using protein from 5-day sheet cultures further confirmed these knockdown results by showing a significantly reduced Hes1 protein expression in shRNA lentivirus-infected MSCs with or without JAG1 treatment (FIG. 4B). More importantly, although the cellular senescence in 5-day cultures was significantly inhibited by JAG1-mediated Notch activation, this inhibitory effect was almost completely abrogated after knocking down Hes1 expression in these MSCs (FIG. 4C). Furthermore, a significantly increased number of SA-β-gal-positive cells in Hes1 shRNA-infected MSCs was observed and quantified by ImageJ (FIG. 4D). Furthermore, real-time PCR data showed an increased gene expression of p16, but not p21, in Hes1 shRNA-infected MSCs (FIG. 4E). Finally, protein expression of p16 in western blot confirmed the inventors' PCR results by showing thinner band in JAG1 treated cells and thicker band in Hes1 deficient cells, and no significant change was observed in protein expression of p21 (FIG. 4F), demonstrating that p16 is one of the most responsible factors for Notch activation-mediated MSC maintenance in cell sheet cultures. Overall, the inventors have demonstrated that Notch inhibition of MSC aging is dependent on the presence of Notch canonical ligand JAG1 and target gene Hes1.

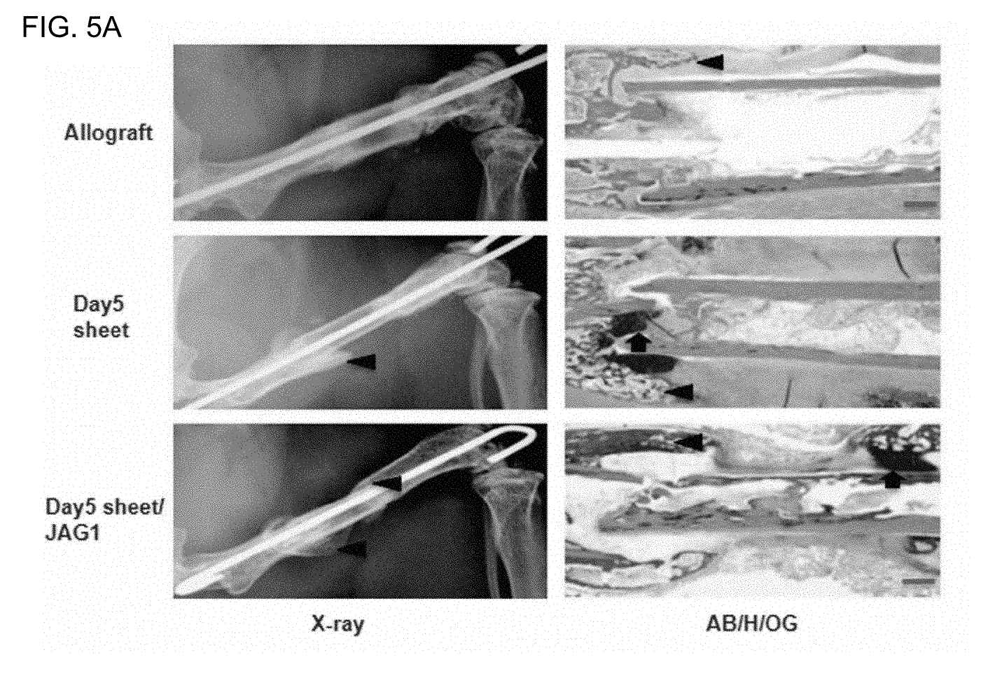

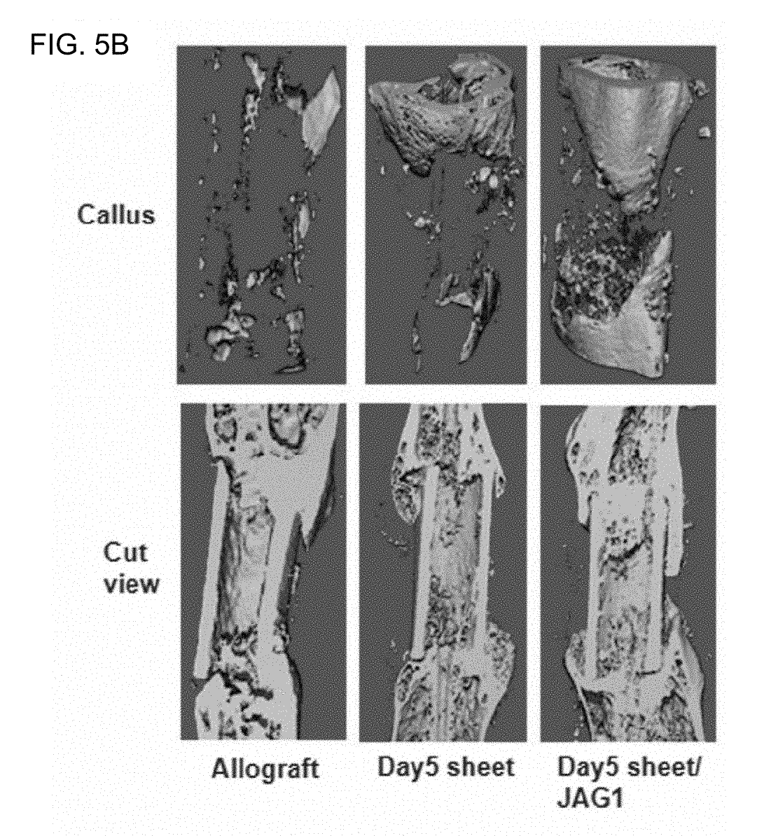

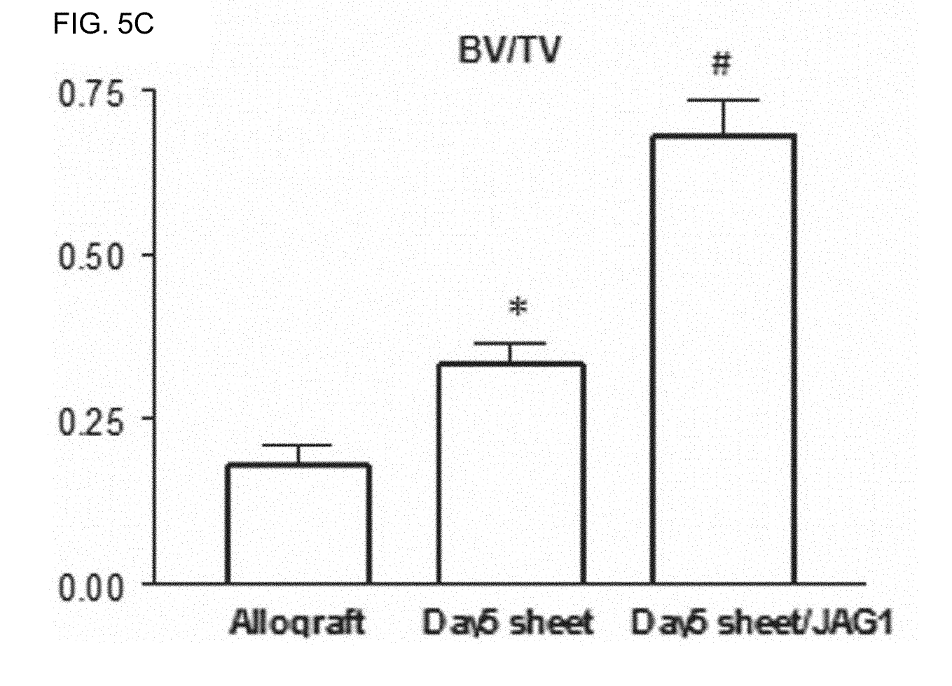

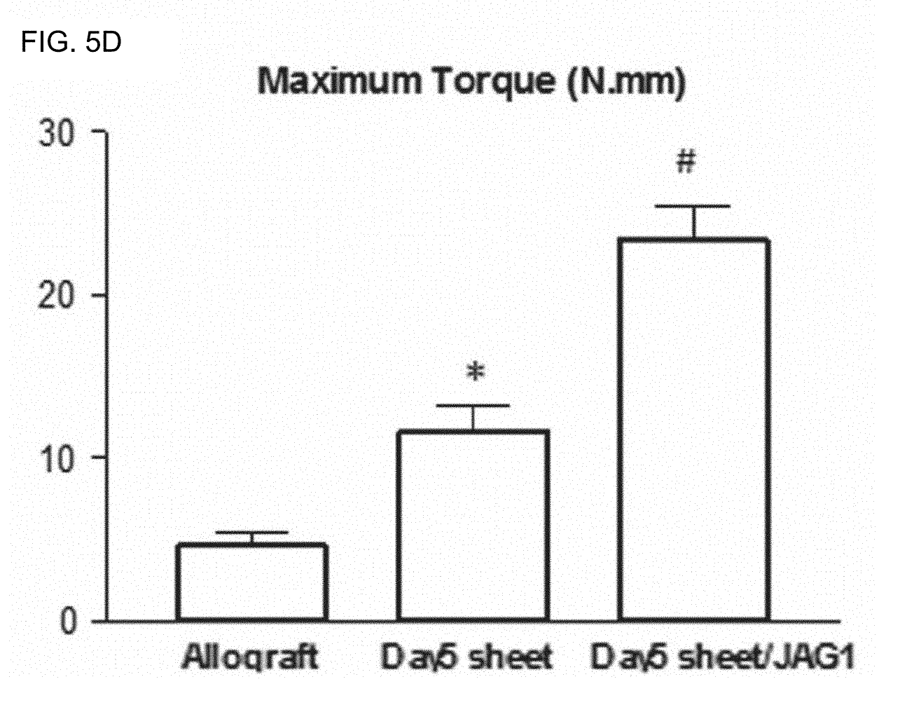

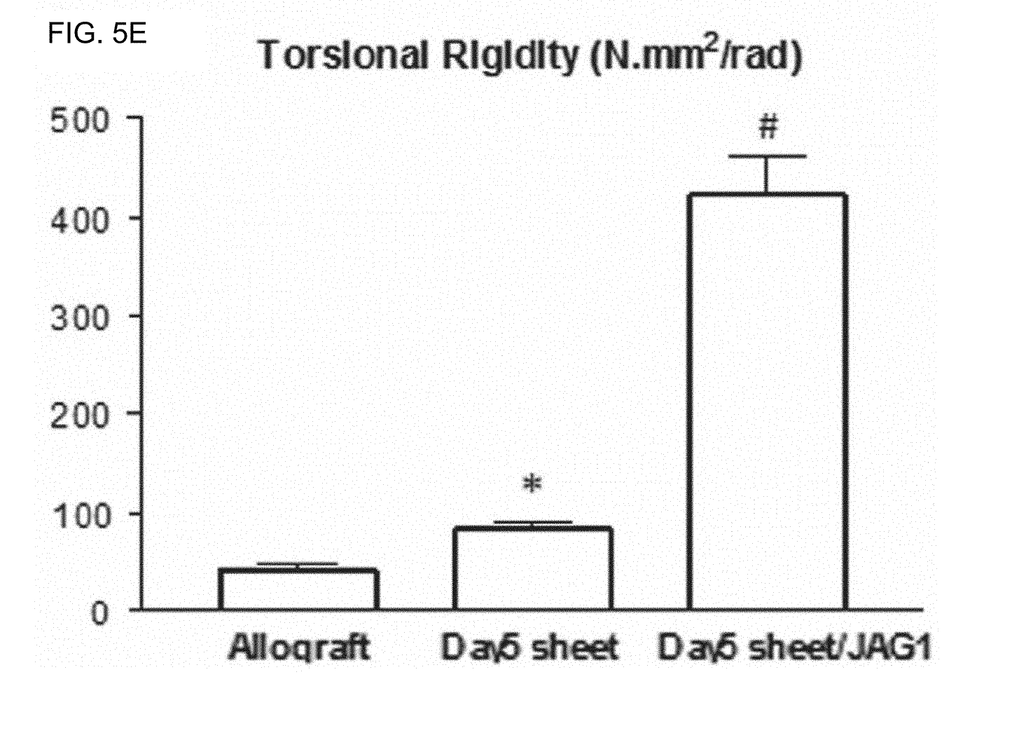

MSC sheets increase bone callus formation and biomechanical properties of femoral allografts: The inventors previously studied the therapeutic effects of 1-day monolayer MSC sheets in a mouse allograft repair model. Here, the inventors further test whether Notch activation-cultured long-term 5-day MSC sheets can also be used in vivo as a pseudo-periosteum to enhance allograft bone defect healing. In these experiments, the inventors transplanted allografts alone, allograft wrapped with IgG-cultured 5-day MSC sheets, or allografts wrapped with Notch activation (JAG1)-cultured 5-day MSC sheets into mouse femoral bone defect models. At six weeks post-surgery, x-ray images (FIG. 5a, left panel) showed a minimal amount of new callus was formed between the allograft and host bone in the allograft alone groups. IgG/5-day MSC sheet-wrapped allografts exhibited large bony callus formation around the proximal allograft and host bone junction, but no significant callus formation was ever observed near the distal allograft and host bone junction. In contrast, a large bridging callus was observed surrounding both ends of allograft in JAG1/5-day MSC-sheet group and the callus formed at proximal gap even reached the mid-allograft surface. AB/H/OG stained sections (FIG. 5A, right panel) confirmed that a large amount of cartilaginous soft callus were still observed at the host/allograft junction in IgG/5-day MSC-sheet groups at 6 weeks, while more bony callus and less cartilaginous soft callus were shown in JAG1/5-day MSC-sheet groups. This suggests a more mineralized bone formed by Notch activation.

To further confirm the inventors' X-ray and histological findings, the inventors performed Micro-CT analysis using samples from 6-weeks post-surgery. Micro-CT analyses (FIG. 5B) demonstrated that the external callus at the host/allograft junction in both MSC sheet groups contained more mineralized bone than the allograft alone groups. However, only JAG1/5-day sheet groups showed a large amount of bony callus formed in the distal end of the allograft gap. The percentage of bone volume (BV) to total tissue volume (TV) of callus was calculated, and identified as significantly higher in the JAG1/5-day MSC-sheet groups when compared to IgG/5-day sheet groups and allograft alone groups (FIG. 5C). Finally, to determine whether increased bone callus formation could be translated into improved mechanical properties, the inventors performed torsional testing on the grafted femurs at 6-weeks post-surgery. Our results showed increased biomechanical properties in IgG/5-day MSC sheet groups were observed when compared with allograft alone groups (FIGS. 5D and 5E). More importantly, the biomechanical strength in JAG1/5-day MSC sheet groups was even higher than that in IgG/5-day groups by showing maximum torque of 22.4 N mm and torsional rigidity of 442.2 N mm²/rad, respectively (FIGS. 5D and 5E).

Discussion: For several years, cell sheet technology using temperature-responsive culture dishes has been applied to tissue engineering to regenerate damaged tissues. As mentioned above, the inventors demonstrated that tissue-engineered periosteum generated with short-term MSC sheet culture enhances callus formation during allograft repair. However, due to the often un-expected transplantation schedule in the clinical settings, long-term cultured MSC sheets should also be studied for their therapeutic efficacy. To monitor MSC maintenance in long-term sheet culture, the inventors first measured the phenotypic change of MSCs in 1-day and 5-day cell sheet cultures. Consistent with previous findings, the inventors observed a progressive cell aging from day 1 to day 5 by showing enhanced cell spontaneous differentiation and senescence. This is in line with cell cycle analysis that cell sheet culture stimulates MSCs cell cycle exit from G1 and S/G2/M phases to the G0 phase, which is an initial step for cell senescence.

As described earlier, senescence is a critical cellular response for continuous aging. Increased senescence often reduces cell proliferation and differentiation potential and impairs tissue regeneration. It is characterized by increased cell cycle arrest in the G0 phase, altered gene expression of growth regulatory proteins (such as p21 and p16), morphologic transformations, and enhanced senescence-associated SA-β-gal activity. In addition, culture senescence limits culture time, thereby preventing cell expansion to the numbers required for cell-based therapies. These observations pose a significant challenge that must be overcome in order to apply cellular therapies in clinical settings. Since both MSC maintenance and lineage differentiation are controlled extrinsically by cellular signals, one of the most promising approaches to prevent MSC ex vivo aging is to target the signaling pathways required for MSC maintenance. Several signaling pathways might be involved in cellular senescence. For example, Ras/Raf/MEK/ERK is reported to be activated in oncogene-induced senescence. Interestingly, the inventors' previous data showed that activation of Notch signaling by JAG1 in MSCs not only promotes cell proliferation but also inhibits cell differentiation to keep cells in a younger stage. These findings led the inventors to test whether Notch signaling could be used to prevent or reverse MSC senescence induced by cell sheet culture. Data from this study clearly support the inventors' hypothesis by showing that JAG1-mediated Notch activation significantly reduced the number of senescence cells in long-term MSC sheet cultures. In addition, less MSCs in G0 phase were observed in JAG1-coated plates confirmed this JAG1 dependent anti-aging effect.

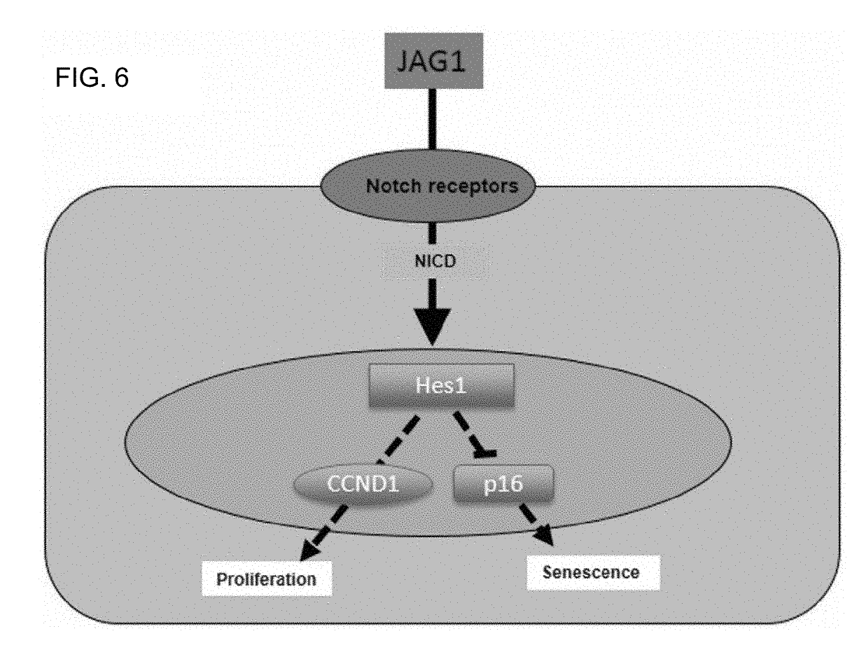

p16 expression is associated with the cellular senescence process through a telomere-dependent or telomere-independent mechanism in most mammalian tissues. With regard to cell cycle checkpoints, p16 controls the G1-S transition by binding to CDK4/6, inhibiting its kinase activity and thereby preventing Rb phosphorylation. The inventors' results further show that Notch activation by JAG1 reduces MSC cellular senescence and decreases expression of p16, but not of p21, in cell sheet cultures, suggesting that p16 plays an important role in JAG1-mediated inhibition of MSC senescence (FIG. 6). The inventors' cell cycle analysis further supported this conclusion by showing reduced G0 phase cell populations in cell sheet cultures with Notch activation by JAG1.

Although the inventors' data indicate that JAG1-mediated Notch signaling alone can partially reverse MSC aging in cell sheet culture, whether it functions through canonical or non-canonical Notch signaling is still unknown. Notch target genes have been identified in various cellular and developmental contexts, including Hes1, Hes5, and Hes7, as well as Hey1, Hey2, and HeyL. As our previous study demonstrated that Hes1 is the most responsive factor in MSCs for JAG1-mediated Notch activation, the inventors further examined the role of Hes1 in JAG1-mediated inhibition of MSC aging. The inventors' loss-of-function experiments clearly show that knockdown of Hes1 expression in MSC sheet culture totally abrogates JAG1-mediated anti-aging effects, suggesting that Hes1 is a or the major molecular responsible for the inhibition of cell senescence (FIG. 6). Further in-depth studies are preferred to understand the mechanistic participation of Hes1-regulated anti-aging effects in cells, including how Hes1 regulates p16 and p21 activity in MSCs.

Since increased MSC senescence often impairs their therapeutic potential in vivo, the inventors further examined the effects of Notch activation cultured 5-day sheets, which contains fewer senescent MSCs on allograft healing. Allograft alone and IgG-cultured 5-day MSC sheets that contain more aging senescence where MSCs were used as controls. Because human MSCs show immune modulation effect and do not induce significant inflammatory immune response in C57BL/6 J mice, the inventors decided to use wild type C57BL/6 J mice in this study to validate the effect of human stem cell sheet on allograft healing. As the inventors expected, the results clearly showed a significant increase of bone callus formation and biomechanical property in Notch activation cultured 5-day sheet groups indicating JAG1-coated plates culture could be utilized to enhance MSC osteogenic potential via inhibition of MSC aging. It should be noted that there were less bone callus formed in IgG-cultured 5-day sheet groups when compared with JAG1-cultured 5-day sheets, but it was significantly more than that found in allograft alone groups suggesting even aged MSC sheet can facilitate bone callus formation. One possible reason is that while the MSCs in long-term cultured sheets are aging, the extracellular matrix surrounding MSCs contains active growth factors that may attract or induce endogenous MSCs migration and differentiation towards osteoblasts in vivo.

Conclusions:

In summary, JAG1-mediated Notch activation was used in this study to prevent MSC aging in long-term sheet cultures and to enhance cell sheet osteogenic potential in vivo. The inventors' study demonstrates that activation of Notch signaling is a promising novel strategy for reversing the aging clock in senescent MSCs and for promoting the appropriate application of Notch signaling to enhance the therapeutic effects of MSCs for regeneration of bone tissue.

Materials and Methods

Animal study design: C57BL/6 J male mice were purchased from Jackson Laboratory. Allogeneic bone grafts were obtained from mice of the 129/J strain for implantation into C57BL/6 J mice. Louisiana State University Committee of Animal Resources approved all animal surgery procedures (Protocol # P-15-005). Experiments were designed to include 12 male mice samples per group. Host mice carrying allografts were randomly and equally assigned to either control (Allograft alone), 5-day IgG-coated plates cultured MSC-sheet or 5-day JAG1-coated plates cultured MSC-sheet groups. The sample size (n=6) for Micro-CT and biomechanical testing and the size (n=6) for histology was determined by power analysis based on our pilot experiment data.

Cultivation of MSCs and lentivirus transduction: MSCs derived from human bone marrow were obtained from Lonza Group Ltd. (http://www.lonza.com). MSCs were first cultured in MSCGM Mesenchymal Stem Cell Growth Medium (Lonza, Cat. PT-3001) until 80% confluence as passage 1 (P1) cells. Further expanded P1 stem cells were harvested as P2 MSCs for different assays as described below. Hes1-specific (shHes1) and control (Co) short hairpin RNA (scrambled shRNA) lentiviral particles were purchased from Sigma-Aldrich. For lentiviral infection, 2000 cell/mm² MSCs were seeded in six-well plates (Nunc, polystyrene) and incubated for 24 h at 37° C. prior to being combined with shRNA lentivirus in the presence of 8 μg/ml polybrene (Sigma-Aldrich). The infected MSC cultures were harvested at day 5 after the cell sheet had formed. Protein expression of Hes1, cell cycle inhibitor p16 and p21 in cells was detected by western blot analysis using antibodies from Santa Cruz and Abcam (Cambridge, Mass., USA), as described previously. Lentiviral GFP control was also used to monitor the infection efficiency.

Recombinant Jagged1-coated culture plates: The human JAG1 recombinant protein (Enzo Life Sciences, Farmingdale, N.Y., USA) contains the signal peptide and extracellular domain of JAG1 fused at the C terminus to the Fc portion of human IgG. JAG1-coated plates were generated using the protocol previously described. Briefly, culture plates (Nunc, polystyrene) were coated with anti-human IgG (10 mg/ml) (Sigma-Aldrich, St. Louis, Mo., USA) in PBS at 4° C. overnight, and then incubated in a solution containing recombinant JAG1 protein (10 mg/ml) at 4° C. overnight. The same concentrations of human IgG were used to coat the plates and the controls. All P2 MSCs were cultured on JAG1- and IgG-coated plates for an additional passage (3-5 days) prior to harvest at days 1 and 5 after forming cell sheets, respectively, for the following assays.

Proliferation and apoptotic assays: P2 MSCs were seeded at 5000 cell/cm² cells in six-well plates (Nunc, polystyrene). At day 1 and day 5 after formation of the cell sheets, the cells were harvested for flow cytometry and proliferation and apoptosis assays. For the proliferation assay, cells were exposed to BrdU-labeling reagent (Roche, Basel, Switzerland) for 6 h, followed by incubation with FixDenat buffer for 30 min, and detection with an anti-BrdU-POD working solution. Absorbance values were measured by a multi-mode microplate reader (BioTek Instruments, Winooski, Vt., USA) at 450 nm. The percentage of apoptotic cells was determined using Apo-ONE homogeneous Caspase-3/7 assay (Promega Biosciences, San Luis Obispo, Calif., USA). Absorbance values were measured by a multi-mode microplate reader at 530 nm, as described before.

Flow cytometry analysis: The MSC phenotype was evaluated by the expression of negative marker CD45-APC (BD Biosciences Pharmingen, San Diego, Calif.) and positive marker CD105-APC (BD Biosciences Pharmingen, San Diego, Calif., USA). P2 Cells in sheet culture were incubated for 30 min at room temperature with antibodies, and flow cytometry was performed on a LSR-II flow cytometer (Beckton Dickson). Isotype-matched IgG-APC controls were included and used to set the electronic gates on the flow cytometer. Cell cycle distribution of MSCs from 1-day and 5-day sheet cultures was determined by flow cytometric analysis after labeling with FITC-conjugated anti-Ki-67 (clone MIB-1; Immunotech, Westbrook, Me., USA) and 5 μg/ml 7-aminoactinomycin-D ([7-AAD] Sigma). All the data were analyzed using FlowJo software (Tree Star).

MSC differentiation assay: MSC spontaneous differentiation assays were performed using standard stem cell growth medium. MSCs were cultured at 5000 cells/cm² to form the cell sheet on standard culture dishes (Nunc, polystyrene) and harvested at days 1 and 5. RNA isolation and ALP staining were performed as previously described.

Senescence-associated-β-galactosidase assay: The senescence-associated-β-galactosidase activity was detected using the SA-β-gal staining kit (Cell Signaling Technology, Boston, Mass., USA) according to the manufacturer's recommendations, except that citric acid/sodium phosphate buffered-staining solution (pH 6.0) was used, as described earlier. Cells were photographed using an EVOS phase-contrast microscope (Advanced Microscopy Group, Bothell, Wash., USA). SA-β-gal-positive cells were counted in five randomly selected fields of view to determine the percentage of β-gal+ cells (>400 cells were counted).

Real time RT-PCR: DNA was synthesized from 1 μg total RNA using the SuperScript III reverse transcriptase kit (Invitrogen, Carlsbad, Calif., USA) in a final volume of 20 μl. Primers were designed with the IDT SCI primer design tool (Integrated DNA Technologies, San Diego, Calif., USA). RT-PCR experiments were performed with a Bio-Rad C1000 thermal cycler (Bio-Rad, Hercules, Calif., USA) in triplicate. The sequences for each primer pair were as follows: Cyclin D1 (CCND1): forward, 5′-ATGGAACATCAGCTGCTGT-3′, and reverse, 5′-TCAGATGTCCACATCCCGC-3′ p21: forward, 5′-GCCTGGACTGTTTTCTCTCG-3′, and reverse, 5′-ATTCAGCATTGTGGGAGGAG-3′ p16: forward, 5′-CACGGGTCGGGTGAGAGT-3′, and reverse, 5′-CCCAACGCACCGAATAGTTAC-3′ alkaline phosphatase (ALP): forward, 5′-GGGCATTGTGACTACCACTC-3′, and reverse, 5′-AGTCAGGTTGTTCCGATTCA-3′ Hes1: forward, 5′-TTCCTCCTCCCCGGTGGCTG-3′, and reverse, 5′-TGCCCTTCGCCTCTTCTCCA-3′ β-actin: forward, 5′-ACCACAGTCCATGCCATCAC-3′, and reverse, 5′-TCCACCACCC TGTTGCTGTA-3′. The relative expression level of target genes was normalized with geNorm software (Primer Design Ltd) using β-actin gene as a reference to determine the normalization factor.

MSC sheets transplantation: Regular cultured 5-day MSC sheets and Notch activation cultured 5-day MSC sheets were generated using IgG or JAG1 coated temperature-responsive plates (Nunc UpCell, Thermo Scientific, Cat. 174901). Total of 36 eight-week-old male C57BL/6 J mice were randomly divided into three groups. Following a surgical procedure, a 4-mm mid-diaphyseal segment bone defect was made, and a 4-mm long allograft wrapped with IgG/5-day sheet or JAG1/5-day sheet was then inserted into the segmental defect and stabilized using a 26-gauge metal pin placed through the intramedullary marrow cavity. Allograft alone was used as the control. Mice were killed at six weeks post-surgery and samples processed for X-ray, Micro-CT bone imaging analyses, biomechanical testing, and histological evaluation with Alcian Blue/Hematoxylin/Orange-G (AB/H/OG) staining as described in our published studies.⁷

Statistical analysis: All experiments were repeated independently at least three times. All data were presented as mean±S.D. Statistical significance among the groups was assessed with one-way ANOVA. The level of significance was P<0.05.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/we claim:

1. A method of retaining multi-potency in stem cells comprising:
one of reducing p16RNA expression and increasing Hes1 expression.

2. The method of claim 1 wherein p16RNA expression is reduced by activating Notch.

3. The method of claim 1 wherein Hes1 expression is increased by activating Notch.

4. The method of claim 1 wherein one of p16RNA expression is reduced by activating Notch and Hes1 expression is increased by activating Notch.

5. The method of claim 4 wherein Notch is activated by adding Jagged1

6. The method of claim 1 wherein the stem cells are mesenchymal stem cells (MSC).

7. The method of claim 1 wherein the stem cells are in a high density sheet.

8. A therapeutic comprising:
a high density sheet of stem cells;
wherein the stem cells are Notch activated.

9. The therapeutic of claim 8 wherein Notch is activated with Jagged1.

10. A method of treating bone injury comprising:
administering stem cells that are Notch activated.

11. The method of claim 10 wherein the stem cells are mesenchymal stem cells (MSC).

12. The method of claim 10 wherein the stem cells are in a high density sheet.

13. The method of claim 10 wherein the stem cells are long term cultured cell sheet.

14. The method of claim 13, wherein the stem cells are cultured at least 5 days.

15. The method of claim 10 wherein the stem cells are part of an allograft administered to the bone injury of the patient.

16. The method of claim 10 wherein the stem cells are mesenchymal stem cells (MSC), the stem cells are in a high density sheet, the stem cells have been cultured for at least 5 days, and the stem cells are part of an allograft administered to the bone injury of the patient.


SEQUENCE LISTING (12 sequences)

SEQ ID NO: 1 - DNA, 19 bp, Homo sapiens
atggaacatcagctgctgt

SEQ ID NO: 2 - DNA, 19 bp, Homo sapiens
tcagatgtccacatcccgc

SEQ ID NO: 3 - DNA, 20 bp, Homo sapiens
gcctggactgttttctctcg

SEQ ID NO: 4 - DNA, 20 bp, Homo sapiens
attcagcattgtgggaggag

SEQ ID NO: 5 - DNA, 18 bp, Homo sapiens
cacgggtcgggtgagagt

SEQ ID NO: 6 - DNA, 21 bp, Homo sapiens
cccaacgcaccgaatagttac

SEQ ID NO: 7 - DNA, 20 bp, Homo sapiens
gggcattgtgactaccactc

SEQ ID NO: 8 - DNA, 20 bp, Homo sapiens
agtcaggttgttccgattca

SEQ ID NO: 9 - DNA, 20 bp, Homo sapiens
ttcctcctccccggtggctg

SEQ ID NO: 10 - DNA, 20 bp, Homo sapiens
tgcccttcgcctcttctcca

SEQ ID NO: 11 - DNA, 20 bp, Homo sapiens
accacagtccatgccatcac

SEQ ID NO: 12 - DNA, 20 bp, Homo sapiens
tccaccaccctgttgctgta